(19) United States
(12) Patent Application Publication
(10) Pub. No.: US 2025/0230209 A1
(43) Pub. Date: Jul. 17, 2025

(54) Isolation of Autologous Acellular Biological Therapeutics

(71) Applicant: GLOBUS MEDICAL, INC., AUDUBON, PA (US)

(72) Inventors: Bipin Gaihre, Spring City, PA (US); Jason Leigl, Collegeville, PA (US); Joshua Cige, Conshohocken, PA (US); Archana Bhat, Phoenixville, PA (US)

(21) Appl. No.: 18/411,162

(22) Filed: Jan. 12, 2024

(51) Int. Cl. C07K 14/49; B01D 61/14; B01D 69/02; C07K 14/545; C07K 14/75; C07K 14/81

(52) U.S. Cl. CPC C07K 14/49; B01D 61/147; B01D 69/02; C07K 14/545; C07K 14/75; C07K 14/81; B01D 2325/02834

(57) ABSTRACT

Various implementations include approaches and systems for isolating acellular biological therapeutics. In a particular implementation, a method includes: drawing leukocyte- and platelet-rich plasma (L-PRP) into a first syringe, passing the L-PRP from the first syringe through a syringe filter to provide filtered L-PRP, drawing the filtered L-PRP into a second syringe, the second syringe having at least one protein precipitating agent (PPA) to provide a L-PRP/PPA mixture, performing a phase separation of protein precipitate and liquid phase from the L-PRP/PPA mixture, removing the liquid phase to yield a concentrated protein precipitate, passing the concentrated protein precipitate and water through a filtering tube, the filtering tube isolating concentrated protein from the concentrated protein precipitate and water, and collecting the concentrated protein.

### TECHNICAL FIELD

This disclosure generally relates to the field of biological therapeutics. More particularly, the disclosure relates to approaches and systems for isolating autologous acellular biological therapeutics.

### BACKGROUND

Degenerative musculoskeletal diseases (DMDs) are a leading cause of pain-associated functional decline, resulting in long-term debilitation. Current first line approaches to treating a cluster of DMDs including but not limited to osteoarthritis (OA), degenerative disc disease (DDD), and osteoporosis, focus on relieving pain through conservative treatments such as physical therapy and over the counter pain medications such as, e.g., non-steroidal anti-inflammatory drugs (NSAIDs). Such conservative treatment modalities have high long-term rates of failure to provide consistent pain relief. This may lead to use of opioids, epidural injections, and eventually surgical solutions when more conservative treatments do not provide consistent pain relief.

Recently, certain orthobiologics derived from both allogenic and autologous sources have shown potential for treatment of DMDs. However, stringent regulatory requirements for manufacturing of allogenic therapies limits accessibility of these treatments to patients, especially for cell and protein therapy products. Clinically, certain autologous biologics have been found attractive. Compared to allogenic therapies, autologous therapies may offer greater convenience, less regulatory stringency, and easier preparation of the final therapeutic product. However, patient outcomes from conventional autologous biologic therapies have been mixed.

### SUMMARY

The needs above, as well as others, are addressed by embodiments of apparatuses for providing feedback on implants, as well as systems for providing implant feedback, and related methods described in this disclosure. All examples and features mentioned below can be combined in any technically possible way.

Various implementations include approaches and systems for isolating acellular biological therapeutics.

In a particular implementation, a method includes: drawing leukocyte-poor and platelet-rich plasma (L-PRP) into a first syringe, passing the L-PRP from the first syringe through a syringe filter to provide filtered L-PRP, drawing the filtered L-PRP into a second syringe, the second syringe having at least one protein precipitating agent (PPA) to provide a L-PRP/PPA mixture, performing a phase separation of protein precipitate and liquid phase from the L-PRP/PPA mixture, removing the liquid phase to yield a concentrated protein precipitate, passing the concentrated protein precipitate and water through a filtering tube, the filtering tube isolating concentrated protein from the concentrated protein precipitate and water, and collecting the concentrated proteins that includes at least one or all of platelet-derived growth factor (PDGF), alpha 2 macroglobulin (α2M), interleukin-1-receptor antagonist protein (IRAP), or fibrinogen.

In another particular implementation, a system for isolating autologous acellular biological therapeutics includes: a first syringe for drawing leukocyte-poor and platelet-rich plasma (L-PRP) from a precursor, a syringe filter for filtering the L-PRP, a second syringe for drawing filtered L-PRP from the syringe filter, mixing the L-PRP with at least one protein precipitating agent (PPA), and facilitating phase separation of protein precipitate and liquid phase from a L-PRP/PPA mixture, a third syringe for providing water, a filtering tube for receiving a concentrated protein precipitate from the second syringe and the water from the third syringe, the filtering tube for isolating concentrated protein from the concentrated protein precipitate, and a collection syringe for collecting the concentrated proteins that includes at least one or all of platelet-derived growth factor (PDGF), alpha 2 macroglobulin (α2M), interleukin-1-receptor antagonist protein (IRAP), or fibrinogen.

In another particular implementation, a system for isolating autologous acellular biological therapeutics includes: a first syringe for drawing leukocyte-poor and platelet-rich plasma (L-PRP) from a precursor, an ultrasonication system to facilitate the lysis of concentrated L-PRP to generate L-PRP lysate (L-PRPL), a syringe filter for filtering the L-PRPL, a second syringe for drawing filtered L-PRPL from the syringe filter, mixing the L-PRPL with at least one protein precipitating agent (PPA), and facilitating phase separation of protein precipitate and liquid phase from a L-PRPL/PPA mixture, a third syringe for providing water, a filtering tube for receiving a concentrated protein precipitate from the second syringe and the water from the third syringe, the filtering tube for isolating concentrated protein from the concentrated protein precipitate, and a collection syringe for collecting the highly concentrated proteins that includes at least one or all of platelet-derived growth factor (PDGF), transforming growth factor beta (TGF-β).

Implementations may include one of the following features, or any combination thereof.

In certain examples, the concentrated protein is provided as a biological therapeutic. In particular examples, the biological therapeutic includes an acellular or non-cellular biological therapeutic.

In particular cases, a method further includes combining the concentrated protein precipitate and the water in a stopcock connector prior to passing through the filtering tube. In certain examples, the water includes sterile water.

In certain aspects, a volume ratio of the water to the concentrated protein precipitate is approximately 1:1.

In some implementations, the filtering tube includes: a filtering membrane; and three ports including: an air injection port, a water collection port, and a concentrated protein collection port, where the concentrated protein is collected via the concentrated protein collection port.

In certain examples, air is injected to the air injection port from an additional syringe, and water is drawn from the water collection port via another syringe.

In particular cases, drawing of the concentrated protein is performed with another syringe, which can be located at a bottom of the filtering tube and in some cases is aided by gravity.

In certain aspects, the phase separation is gravity-induced.

In particular implementations, the phase separation is performed with the second syringe in an upright position for a period.

In some cases, the period is approximately 5 minutes to approximately 15 minutes.

In certain implementations, after the phase separation, the liquid phase overlies the protein precipitate in the upright position. In some examples, the liquid is mostly water, such as greater than 50% water.

In particular aspects, the first syringe includes a luer lock syringe, and the second syringe includes a precipitation syringe. In some examples, the second syringe is of a distinct type than the first syringe. In particular examples, the second syringe is larger than the first syringe.

In some cases, the method further includes performing ultrasonication of a L-PRP collection to yield the PRP lysate (L-PRPL) prior to drawing the L-PRPL into the first syringe.

In particular cases, ultrasonication includes transfer of concentrated L-PRP derived from the PRP kit to approximately 15 ml or approximately 50 ml conical tubes. In certain examples, the conical tubes are transferred to an ice bath to prevent protein denaturation due to heat generated during ultrasonication. In still further implementations, a disposable ultrasonication probe is inserted in the tube (e.g., while in the ice batch), and L-PRP is sonicated at high speed for approximately 1 min to approximately 15 min to yield the L-PRPL.

In certain examples, the L-PRP collection is obtained from the autologous biologics PRP kit that includes patient specific plasma.

In particular cases, ultrasonication is performed using: an ultrasonication system for increasing protein quantity of the L-PRP collection through the lysis of platelets presents in L-PRP, and a protein precipitation and filtration system, where the L-PRP collection is obtained from an autologous biologics PRP kit.

In some aspects, the syringe filter has a pore size ranging from approximately 0.2 micrometers to approximately 5 micrometers, the PPA includes at least one of: ammonium sulfate, 4-arm polyethylene glycol (aPEG), 8-aPEG, or trichloroacetic acid (TCA) with sodium chloride, and a volume ratio of L-PRP/L-PRPL to PPA is approximately 1:1, approximately 2:1, approximately 3:1, or approximately 3:2.

In one example, the concentration of aPEG to be used as PPA is approximately 5 percent to approximately 10 w/v percent. In further examples, the PPA includes ammonium sulfate, with a concentration of sodium chloride in TCA from approximately 0.05 M to approximately 10 M. In one example, 60 ml of L-PRP/L-PRPL is injected to 30 ml of saturated ammonium sulfate solution, or 20 ml aPEG solution in a precipitation syringe for the precipitation of proteins in the L-PRP/L-PRPL.

In certain cases, in the system, the filtering tube includes a filtering membrane and three ports including: an air injection port, a water collection port, and a concentrated protein collection port. In some cases, the collection syringe is configured to couple with the concentrated protein collection port.

In particular aspects, the air injection port is configured to couple with an air introduction syringe and the water collection port is configured to couple with a water collection syringe.

In some cases, the system further includes: an ultrasonication system for increasing protein quantity of a PRP collection to yield the L-PRPL, and a protein precipitation and filtration system. The ultrasonication system is configured to at least partially cause lysis of concentrated platelets of PRP from the PRP collection.

Two or more features described in this disclosure, including those described in this summary section, may be combined to form implementations not specifically described herein.

The above presents a simplified summary in order to provide a basic understanding of some aspects of the claimed subject matter. This summary is not an extensive overview. It is not intended to identify key or critical elements or to delineate the scope of the claimed subject matter. Its sole purpose is to present some concepts in a simplified form as a prelude to the more detailed description that is presented later.

The details of one or more implementations are set forth in the accompanying drawings and the description below. Other features, objects and benefits will be apparent from the description and drawings, and from the claims.

### BRIEF DESCRIPTION OF THE DRAWINGS

FIG. 1 is a schematic view of a portion of a system for isolating autologous acellular biological therapeutics according to various implementations.

FIG. 2 illustrates a portion of a process for isolating autologous acellular biological therapeutics according to various implementations.

FIG. 3 is a schematic view of an additional portion of a system for isolating autologous acellular biological therapeutics according to various implementations.

FIG. 4 is a schematic view of an optional, additional portion of a system for isolating autologous acellular biological therapeutics according to various implementations.

FIG. 5 is a flow diagram illustrating processes in a method according to various implementations.

It is noted that the drawings of the various implementations are not necessarily to scale. The drawings are intended to depict only typical aspects of the disclosure, and therefore should not be considered as limiting the scope of the implementations. In the drawings, like numbering represents like elements between the drawings.

### DETAILED DESCRIPTION

Various example embodiments of approaches and systems for isolating autologous acellular biological therapeutics are described herein. In the interest of clarity, not all features of an actual implementation are necessarily described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure. The methods, apparatuses and related systems and methods described herein boast a variety of inventive features and components that warrant patent protection, both individually and in combination.

It is understood that any given elements of the disclosed embodiments of the invention may be embodied in a single structure, a single step, a single substance, or the like. Similarly, a given element of the disclosed embodiment may be embodied in multiple structures, steps, substances, or the like.

Commonly labeled components in the FIGURES are considered to be substantially equivalent components for the purposes of illustration, and redundant discussion of those components is omitted for clarity.

Additional aspects, advantages and/or other features of example embodiments of the invention will become apparent in view of the following detailed description. It should be apparent to those skilled in the art that the described embodiments provided herein are merely exemplary and illustrative and not limiting. Numerous embodiments of modifications thereof are contemplated as falling within the scope of this disclosure and equivalents thereto. In describing example embodiments, specific terminology is employed for the sake of clarity. However, the embodiments are not intended to be limited to this specific terminology. Unless otherwise noted, technical terms are used according to conventional usage.

As noted herein, for DMD therapies, certain clinical studies have reported improvements in patient outcomes while others report no improvement at all. The variability in patient-reported outcomes may be attributable to the quality of the biologics, but conventional approaches for developing such biologics have been lacking in one or more aspects.

Various disclosed implementations relate to the development of non-cellular or acellular biological therapeutics derived from blood tissue of the patient, i.e., autologous non-cellular or acellular biological therapeutics, and an accompanying kit that facilitates this isolation. Various disclosed therapeutics may be acellular, e.g., may be completely acellular, but include highly potent regenerative proteins or biomacromolecules which are secreted by the cells. These therapeutics address current variabilities in clinical outcomes, while improving patient conditions. In various particular implementations, the biological therapeutics include concentrated proteins and other biomacromolecules.

Various implementations include approaches and systems to concentrate regenerative proteins of platelet-rich plasma (PRP), for example, to yield isolated autologous acellular biological therapeutics. Various particular implementations include systems and approaches for producing biological therapeutics from leukocyte-poor and platelet-rich plasma (L-PRP). As described herein, the L-PRPL can be derived from L-PRP, e.g., via an ultrasonication approach. However, other approaches for providing L-PRP can be suitably substituted for ultrasonication according to various implementations.

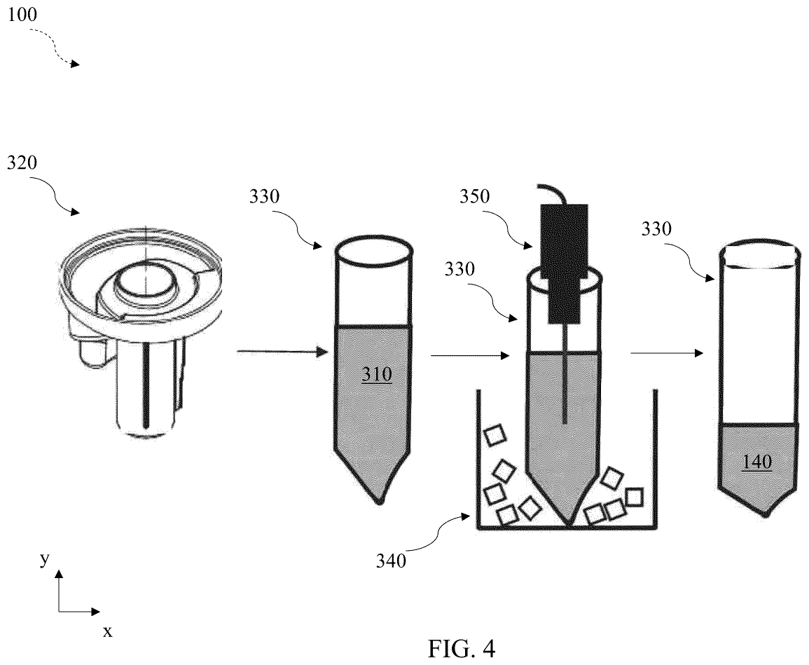

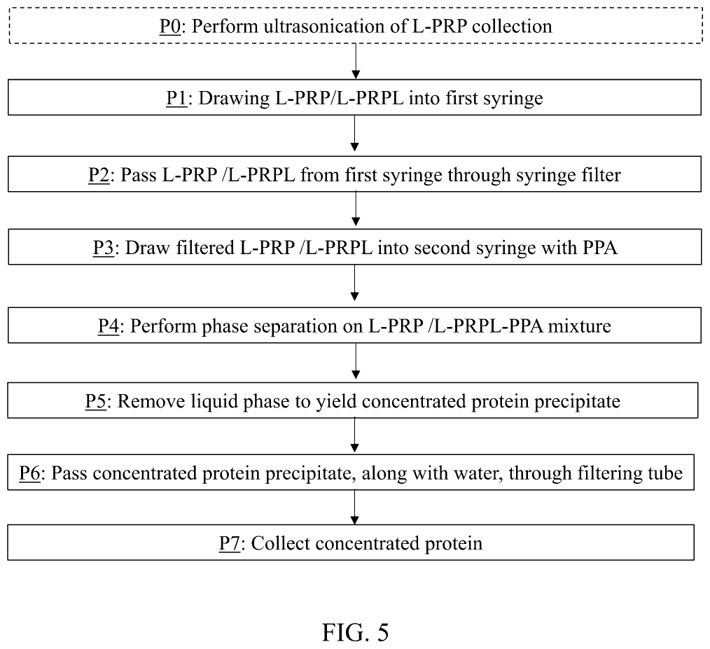

FIGS. 1-3 illustrate portions of a system 100 for isolating autologous acellular biological therapeutics according to various implementations. FIG. 4 shows an optional additional portion of the system 100 for performing ultrasonication on PRP to yield L-PRPL. FIG. 5 is a flow diagram illustrating a method for isolating autologous acellular biological therapeutics according to various implementations.

Turning to FIG. 1, a first portion of the system 100 is illustrated, including a first syringe 110, a second syringe 120, and a syringe filter 130. In certain cases, the first syringe 110 and second syringe 120 differ in at least one aspect. In some examples, the first syringe 110 includes a luer lock syringe. In further examples, the second syringe 120 includes a precipitation syringe. In particular examples, the second syringe 120 is larger than the first syringe 110. In some aspects, the syringe filter 130 has a pore size ranging from approximately 0.2 micrometers to approximately 5 micrometers.

With reference to FIGS. 1 and 5, in a first process (P1, FIG. 5), a method includes drawing L-PRP 140 into the first syringe 110. In certain cases, the L-PRP 140 is drawn from a vial or other holder by the first syringe 110. In a second process (P2, FIG. 5), the L-PRP is passed from the first syringe 110 through the syringe filter 130, to provide filtered L-PRP. In a third process (P3, FIG. 5), the filtered L-PRP is drawn into the second syringe 120. In various implementations, the second syringe 120 contains at least one protein precipitating agent (PPA) prior to drawing the filtered L-PRP, and after drawing the L-PRP from the syringe filter 130, a L-PRP/PPA mixture 150 is produced. In certain examples, the PPA includes at least one of: ammonium sulfate, 4-arm polyethylene glycol (aPEG), 8-aPEG, or trichloroacetic acid (TCA) with sodium chloride, and a volume ratio of L-PRP to PPA is approximately 1:1, approximately 2:1, approximately 3:1, or approximately 3:2. In one example, aPEG is approximately 5 percent to approximately 10 percent w/v. In further examples, the PPA includes ammonium sulfate, with a concentration of sodium chloride in TCA from approximately 0.05M to approximately 10 M. In one example, 60 ml of L-PRP is injected into 30 ml of saturated ammonium sulfate solution, or 20 ml aPEG solution in a precipitation syringe for the precipitation of proteins in the L-PRP.

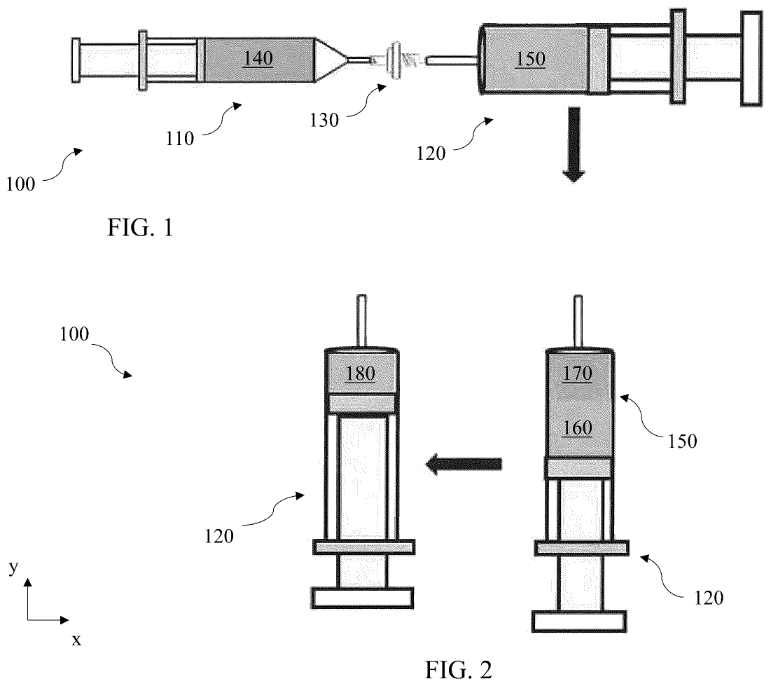

With reference to the right-hand portion of FIG. 2, in a fourth process (P4, FIG. 5), phase separation is performed on the L-PRP/PPA mixture 150 to separate a protein precipitate 160 and liquid phase 170 from the L-PRP/PPA mixture 150. In some examples, as shown in FIG. 2, the phase separation is gravity-induced. For example, phase separation can be performed with the second syringe 120 in an upright position (e.g., with syringe tip pointed upward) for a period. During phase separation, the second syringe 120 can be suspended manually, e.g., by an operator, or by an apparatus (not shown). In certain examples, the period is approximately 5 minutes to approximately 15 minutes. As illustrated in the example depiction in FIG. 2, after the phase separation, the liquid phase 170 overlies the protein precipitate 160 in the upright position. In certain examples, the liquid in the liquid phase 170 is mostly water, e.g., at least 50 percent water, at least 60 percent water, at least 70 percent water, or at least 80 percent water. In an additional process (P5, FIG. 5) shown in the left-hand portion of FIG. 2, the liquid phase 170 is removed (e.g., ejected and/or collected) to yield a concentrated protein precipitate 180.

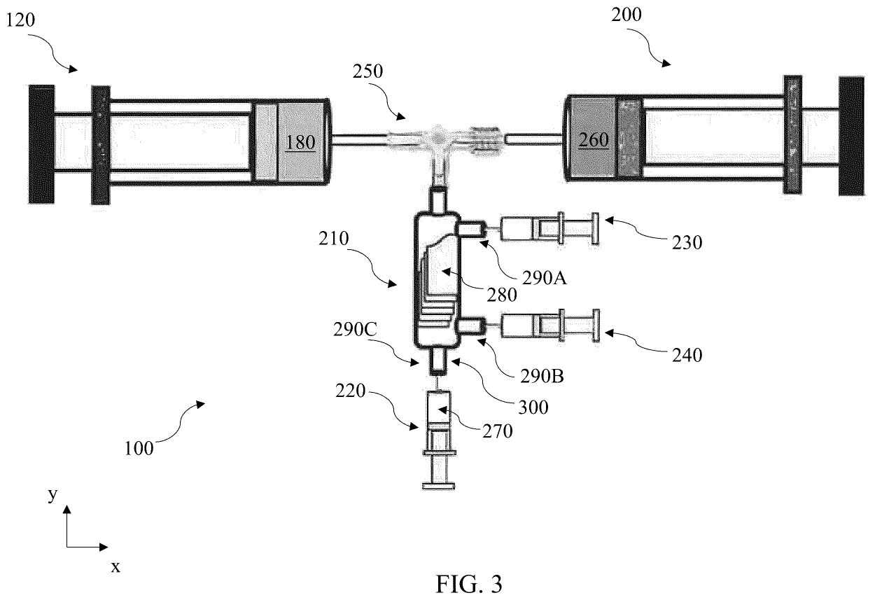

FIG. 3 illustrates an additional set of processes in the method according to various implementations. These processes are illustrated in conjunction with further portions of a system 100, e.g., including a third syringe 200, a filtering tube 210, and a collection syringe 220. In certain cases, as described herein, the system 100 can further include an air injection syringe 230 and a water collection syringe 240. In a particular example, a connector 250 such as a multi-way (e.g., three-way) stopcock connector is used to couple two or more syringes (e.g., second syringe 120 and third syringe 200) and the filtering tube 210. In one example illustrated in FIG. 3, the connector 250 includes a three way stopcock connector. In the depicted example, second syringe 120 is shown including the concentrated protein precipitate 180 from the left-hand portion of FIG. 2. The connector 250 is connected with the filtering tube 210 and the third syringe 200, which includes water 260. In certain examples, the water includes sterile water.

In a further process (P6, FIG. 5), the concentrated protein precipitate 180 is passed, along with water 260 from the third syringe 200, through the filtering tube 210. In certain aspects, a volume ratio of the water to the concentrated protein precipitate is approximately 1:1. According to various implementations, the filtering tube 210 can isolate concentrated protein 270 from the mixture of concentrated protein precipitate 180 and water 260. In another process (P7, FIG. 5), the concentrated protein 270 is collected, for example, in the collection syringe 220.

In some implementations, the filtering tube 210 includes: at least one filtering membrane 280; and three ports 290. In certain cases, a plurality of filtering membranes 280 are included in the filtering tube 210. In particular aspects, the ports 290 include: an air injection port 290A, a water collection port 290B, and a concentrated protein collection port 290C. As shown in FIG. 3, the concentrated protein 270 is collected at the collection syringe 220 via the concentrated protein collection port 290C. In certain examples, air is injected to the air injection port 290A from an additional syringe, e.g., the air injection syringe 230, and water is drawn from the water collection port 290B via another syringe, e.g., the water collection syringe 240. It is understood that naming of particular ports and/or syringes is only illustrative, and not intended to limit the use of those ports and/or syringes for particular purposes. In certain cases, the location(s) of one or more ports and type(s) of one or more syringes may differ from those disclosed and remain in keeping with the disclosed implementations.

In particular cases, the concentrated protein 270 is drawn from a lower, or lowermost port in the filtering tube 210. For example, the concentrated protein collection port 290C can be located at a bottom 300 of the filtering tube 210 when the second 120 and third 200 syringes face one another, or are aligned, along the x-axis. In particular cases, drawing the concentrated protein 270 at the collection syringe 220 is aided by gravity, e.g., where the concentrated protein collection port 290C is below the air injection port 290A and the water collection port 290B along the y-axis.

In certain examples, the concentrated protein 270 is provided as a biological therapeutic. In particular examples, the biological therapeutic 270 includes an acellular or non-cellular biological therapeutic, and may particularly include one or more of platelet-derived growth factor (PDGF), alpha 2 macroglobulin (α2M), interleukin-1-receptor antagonist protein (IRAP), transforming growth factor beta (TGF-β), and fibrinogen.

As noted herein, in certain aspects, the L-PRP drawn into the first syringe 110 (P1, FIG. 5; FIG. 1) is derived from a PRP collection. For example, as illustrated in FIG. 5, a preliminary process (P0) can include performing ultrasonication of a PRP collection to yield the L-PRPL 140 prior to drawing the L-PRP 140 into the first syringe 110. Turning to FIG. 4, ultrasonication can include transferring concentrated L-PRP 310 derived from a PRP kit 320 to tubes 330 (e.g., conical tubes such as approximately 15 ml conical tubes or approximately 50 ml conical tubes). In certain examples, the tubes 330 are transferred to an ice bath 340 to prevent protein denaturation due to heat generated during ultrasonication. In still further implementations, an ultrasonication probe 350 is inserted in the tube (e.g., while in the ice bath 340), and concentrated PRP 310 is sonicated at high speed for approximately 1 min to approximately 15 min to lyse the concentrated platelets of the PRP 310 to release biomacromolecules such as proteins and yield the L-PRPL 140. In particular examples, the ultrasonication probe 350 is disposable, or otherwise configured for single use. In other cases, the ultrasonication probe 350 can be configured for reuse.

In certain examples, the PRP collection (or, concentrated PRP) 310 is obtained from the autologous biologics PRP kit 320 that includes patient specific plasma. An example autologous biologics PRP kit is the Harvest® SmartPrep® 3 System provided by Globus Medical, Inc., Audubon, PA (USA). In certain cases, the autologous biologics PRP kit 320 is an autologous biologic centrifuge platform that generates highly concentrated platelet-rich plasma (PRP), and in further cases, can generate bone marrow aspirate concentrate (BMAC®), and adipose-derived (AdiPrep®) cellular biologics. Registered trademarks shown herein belong to Globus Medical, Inc. Audubon, PA (USA).

In particular cases, ultrasonication increases the protein quantity of the PRP collection 330. In various implementations, the PRP collection 310 is obtained from the autologous biologics PRP kit 320, and includes at least one of platelet-derived growth factor (PDGF), alpha 2 macroglobulin (α2M), interleukin-1-receptor antagonist protein (IRAP), transforming growth factor beta (TGF-β), or fibrinogen. In this optional process (P0), ultrasonication at least partially causes lysis of concentrated platelets of L-PRP from the PRP collection 310.

As noted herein, the various disclosed approaches and systems can effectively concentrate regenerative proteins of platelet-rich plasma (PRP), for example, to yield isolated autologous acellular biological therapeutics. Various particular implementations include systems and approaches for producing biological therapeutics from a leukocyte-poor and platelet-rich plasma (L-PRP). As described herein, in certain cases, the L-PRP can be derived from PRP, e.g., via an ultrasonication approach. The disclosed approaches and systems can provide an autologous solution for producing acellular biological therapeutics. Further, disclosed approaches that include the lysis of platelets can yield high concentrations of regenerative and high-potency proteins. The disclosed systems can provide precipitation and purification of proteins in a single-module approach. In still further implementations, therapeutics derived from the systems and approaches can minimize variability in patient outcomes. In various implementations, proteins can be directly loaded into injection syringes during purification for easy injection to patients.

Certain additional aspects of producing isolated autologous acellular biological therapeutics are included in U.S. Pat. Nos. 9,399,226; 8,282,839; and 9,138,508, along with US Patent Application Publication No. 2022/0008615, the entire contents of each of which is incorporated by reference herein.

The term “approximately” as used with respect to values herein can allot for a nominal variation from absolute values, e.g., of several percent or less. Where the term “comprising” is used in the present description and claims, it does not exclude other elements or operations. The term “based on” (as in “A is based on B”) is used to indicate any of its ordinary meanings, including the cases (i) “based on at least” (e.g., “A is based on at least B”) and, if appropriate in the particular context, (ii) “equal to” (e.g., “A is equal to B”). Similarly, the term “in response to” is used to indicate any of its ordinary meanings, including “in response to at least.”

As used herein and in the claims, the terms “comprising” and “including” are inclusive or open-ended and do not exclude additional unrecited elements, compositional components, or method steps. Accordingly, the terms “comprising” and “including” encompass the more restrictive terms “consisting essentially of” and “consisting of.”

Unless specified otherwise, all values provided herein include up to and including the endpoints given, and the values of the constituents or components of the compositions are expressed in weight percent or % by weight of each ingredient in the composition.

Each compound used herein may be discussed interchangeably with respect to its chemical formula, chemical name, abbreviation, etc. For example, PEG may be used interchangeably with polyethylene glycol.

Various implementations are described in terms of systems and approaches for isolating autologous acellular biological therapeutics. It is understood that such systems and approaches can be automated or otherwise aided by use of a machine including a programmable processor, functions of which can be implemented, at least in part, via a computer program product, e.g., a computer program tangibly embodied in an information carrier, such as one or more non-transitory machine-readable media, for execution by, or to control the operation of, one or more data processing apparatus, e.g., a programmable processor, a computer, multiple computers, and/or programmable logic components.

In various implementations, components described as being “coupled” to one another can be joined along one or more interfaces. In some implementations, these interfaces can include junctions between distinct components, and in other cases, these interfaces can include a solidly and/or integrally formed interconnection. That is, in some cases, components that are “coupled” to one another can be simultaneously formed to define a single continuous member. However, in other implementations, these coupled components can be formed as separate members and be subsequently joined through known processes (e.g., engaging quick connect couplings, snap-to-fit couplings, twist-to-fit couplings, syringe-to-syringe couplings, syringe-to-filter couplings, etc.). Additionally, sub-components within a given component can be considered to be linked via conventional pathways, which may not necessarily be illustrated.

A number of implementations have been described. Nevertheless, it will be understood that additional modifications may be made without departing from the scope of the inventive concepts described herein, and, accordingly, other implementations are within the scope of the following claims.

We claim:

1. A method comprising:
drawing leukocyte- and platelet-rich plasma (L-PRP) into a first syringe,
passing the L-PRP from the first syringe through a syringe filter to provide filtered L-PRP,
drawing the filtered L-PRP into a second syringe, the second syringe having at least one protein precipitating agent (PPA) to provide a L-PRP/PPA mixture,
performing a phase separation of protein precipitate and liquid phase from the L-PRP/PPA mixture,
removing the liquid phase to yield a concentrated protein precipitate,
passing the concentrated protein precipitate and water through a filtering tube, the filtering tube isolating concentrated protein from the concentrated protein precipitate and water, and
collecting the concentrated protein that includes at least one or all of platelet-derived growth factor (PDGF), alpha 2 macroglobulin (α2M), interleukin-1-receptor antagonist protein (IRAP), or fibrinogen.

2. The method of claim 1, wherein the concentrated protein is provided as a biological therapeutic.

3. The method of claim 1, further comprising combining the concentrated protein precipitate and the water in a stopcock connector prior to passing through the filtering tube.

4. The method of claim 3, wherein a volume ratio of the water to the concentrated protein precipitate is approximately 1:1.

5. The method of claim 1, wherein the filtering tube includes:
a filtering membrane; and
three ports including: an air injection port, a water collection port, and a concentrated protein collection port,
wherein the concentrated protein is collected via the concentrated protein collection port.

6. The method of claim 1, wherein the phase separation is gravity-induced.

7. The method of claim 6, wherein the phase separation is performed with the second syringe in an upright position for a period.

8. The method of claim 7, wherein the period is approximately 5 minutes to approximately 15 minutes.

9. The method of claim 7, wherein after the phase separation, the liquid phase overlies the protein precipitate in the upright position.

10. The method of claim 1, wherein the first syringe comprises a luer lock syringe, and wherein the second syringe comprises a precipitation syringe.

11. The method of claim 1, further comprising performing ultrasonication of a L-PRP collection to yield the L-PRPL prior to drawing the L-PRPL into the first syringe.

12. The method of claim 11, wherein the ultrasonication is performed using:
an ultrasonication system for increasing protein quantity of the PRP collection, and
a protein precipitation and filtration system,
wherein the PRP collection is obtained from an autologous biologics PRP kit, and wherein the PRP collection includes at least one of platelet-derived growth factor (PDGF), alpha 2 macroglobulin (α2M), interleukin-1-receptor antagonist protein (IRAP), or fibrinogen, and
wherein ultrasonication at least partially causes lysis of concentrated platelets of PRP from the PRP collection.

13. The method of claim 1, wherein the syringe filter has a pore size ranging from approximately 0.2 micrometers to approximately 5 micrometers,
wherein the PPA includes at least one of: ammonium sulfate, 4-arm polyethylene glycol (aPEG), 8-aPEG, or trichloroacetic acid with sodium chloride, and
wherein a volume ratio of L-PRP to PPA is approximately 1:1, approximately 2:1, approximately 3:1, or approximately 3:2.

14. A system for isolating autologous acellular biological therapeutics, the system comprising:
a first syringe for drawing leukocyte- and platelet-rich plasma (L-PRP) from a precursor,
a syringe filter for filtering the L-PRP,
a second syringe for drawing filtered L-PRP from the syringe filter, mixing the L-PRP with at least one protein precipitating agent (PPA), and facilitating phase separation of protein precipitate and liquid phase from a L-PRP/PPA mixture,
a third syringe for providing water,
a filtering tube for receiving a concentrated protein precipitate from the second syringe and the water from the third syringe, the filtering tube for isolating concentrated protein from the concentrated protein precipitate, and
a collection syringe for collecting the concentrated protein.

15. The system of claim 14, wherein the concentrated protein is adapted for use as a biological therapeutic.

16. The system of claim 14, wherein the filtering tube includes a filtering membrane and three ports including: an air injection port, a water collection port, and a concentrated protein collection port,
wherein the collection syringe is configured to couple with the concentrated protein collection port.

17. The system of claim 16, wherein the air injection port is configured to couple with an air introduction syringe and wherein the water collection port is configured to couple with a water collection syringe.

18. The system of claim 14, wherein the first syringe comprises a luer lock syringe, and wherein the second syringe comprises a precipitation syringe.

19. The system of claim 14, further comprising:
an ultrasonication system for increasing protein quantity of a PRP collection to yield the L-PRP, and
a protein precipitation and filtration system,
wherein the ultrasonication system is configured to at least partially cause lysis of concentrated platelets of PRP from the PRP collection.

20. The system of claim 14, wherein the syringe filter has a pore size ranging from approximately 0.2 micrometers to approximately 5 micrometers,
wherein the PPA includes at least one of: ammonium sulfate, 4-arm polyethylene glycol (aPEG), 8-aPEG, or trichloroacetic acid with sodium chloride, and
wherein a volume ratio of L-PRP to PPA is approximately 1:1, approximately 2:1, approximately 3:1, or approximately 3:2.
